(19) United States
(12) Patent Application Publication
(10) Pub. No.: US 2024/0058375 A1
(43) Pub. Date: Feb. 22, 2024

(54) CO-FORMULATION OF POLYMYXINS FOR INHALATION

(71) Applicant: Purdue Research Foundation, West Lafayette, IN (US)

(72) Inventors: Qi ZHOU, West Lafayette, IN (US); Maizbha Uddin AHMED, West Lafayette, IN (US); Mohammad Abul Kalam AZAD, Melton South (AU); Jian LI, Carnegie, Victoria (AU)

(73) Assignee: Purdue Research Foundation, West Lafayette, IN (US)

(21) Appl. No.: 18/260,190

(22) PCT Filed: Oct. 27, 2021
(86) PCT No.: PCT/US2021/056727

§ 371 Date: Jun. 30, 2023

Related U.S. Application Data
(60) Provisional application No. 63/133,257, filed on Jan. 1, 2021.

(51) Int. Cl. A61K 31/785; A61K 38/12; A61K 9/00

(52) U.S. Cl. CPC A61K 31/785; A61K 38/12; A61K 9/0073; A61K 9/0075

(57) ABSTRACT

The present disclosure generally relates to a method for reducing the toxicity of polymyxins as a therapeutic agent comprising the step of coadministration of an aminoglycoside; a method for improving the aerosolization of an aminoglycoside comprising the step of combination formulation with a polymyxin; and a process for manufacturing a dry powder composition comprising a polymyxin and aminoglycoside. Pharmaceutical compositions and methods of treatment for lung infections are within the scope of this invention.

### CROSS REFERENCE TO RELATED APPLICATIONS

This present patent application is related to and claims the priority benefit of U.S. Provisional Patent Application Ser. No. 63/133,257, filed Jan. 1, 2021, the contents of which are hereby incorporated by reference in their entirety into this disclosure.

### GOVERNMENT SUPPORT CLAUSE

This invention was made with government support under AI132681 awarded by the National Institutes of Health. The government has certain rights in the invention.

### TECHNICAL FIELD

The present application generally relates to a method for reduced toxicity and improved therapeutic efficacy of polymyxins comprising the step of co-formulation of polymyxins with polyaspartic acid; and a process for manufacturing solution, suspension or dry powder composition comprising polymyxins and polyaspartic acid for pulmonary delivery. Pharmaceutical compositions and methods of treatment for lung infections are within the scope of this invention.

### BACKGROUND

This section introduces aspects that may help facilitate a better understanding of the disclosure. Accordingly, these statements are to be read in this light and are not to be understood as admissions about what is or is not prior art.

Inhaled polymyxins have been used to treat pulmonary infections caused by Gram-negative ‘superbugs’ (e.g. Pseudomonas aeruginosa, Acinetobacter baumannii and Klebsiella pneumoniae) over the last decades.¹⁻⁴ However, the dosage regimens are empirical and have never been optimized using pharmacokinetic/pharmacodynamic principles. High doses of inhaled polymyxins may cause pulmonary toxicities, such as pulmonary eosinophilia, hypersensitivity pneumonitis and acute respiratory failure. ⁵⁻⁸ Our recent studies demonstrated that polymyxins may cause apoptosis in lung epithelial cells and involved oxidative stress and mitochondrial toxicity such as mitochondrial membrane depolarization. ⁹ Our imaging study also revealed that polymyxins significantly co-localize with mitochondria in lung epithelial cells. ¹⁰ Furthermore, untargeted metabolomics results showed that polymyxin treatment significantly affected multiple pathways in lung epithelial cells including membrane phospholipids biosynthesis.¹¹ Therefore, it is imperative to develop novel approaches to minimize polymyxin-induced pulmonary toxicity.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIGS. 1A-1E. Inhibition of polymyxin B induced cellular toxicity in A549 cells by polyaspartic acid at 24 h. PAA, Polyaspartic acid; PMB, polymyxin B. Data are presented as mean±SD (n=3). Attenuation of polymyxin B induced toxicity in A549 cells by polyaspartic acid at 24 h. FIGS. 1A-1B: attenuation of cell death; FIG. 1C: attenuation of mitochondrial superoxide formation; FIG. 1D: restoration of mitochondrial membrane potential; and FIG. 1E: inhibition of intracellular polymyxin B uptake. PAA, Polyaspartic acid; PGA, polyglutamic acid, PMB, polymyxin B. Data are presented as mean±SD (n=3).

FIG. 2A: Histological assessment of the magnitude of lung tissue injury in different treatment groups. Scale bar=100 μm (×10 magnification). FIG. 2B: Combination of polymyxin B and polyaspartic acid showed significant reduction in the SQS assessed by polymyxin B alone (p<0.01). Data are presented as mean±SD (n=3).

FIGS. 3A-3E. Representative scanning electron microscopy (SEM) images of spray-dried pure colistin (FIG. 3A), pure polyaspartic acid (FIG. 3B), and their combination formulations at various ratios: 5:1 (FIG. 3C); 1:1 (FIG. 3D); and 1:5 (FIG. 3E).

FIG. 4. X-ray diffraction patterns of different spray-dried formulations

FIGS. 5A-5C. FPF (FIG. 5A), ED (FIG. 5B), and E-FPF (FIG. 5C) values of spray-dried formulations of colistin and polyaspartic acid (mean±SD, n=4). * significantly different from SD pure col with p<0.05, ** p<0.01 and *** p<0.001.

FIG. 6. NGI deposition profiles of spray-dried formulations of colistin and polyaspartic acid (mean±SD, n=4). * Significantly different from SD pure col with p*<0.05, ** p<0.01 and *** p<0.001, **** p<0.0001.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 20%, within 10%, within 5%, or within 1% of a stated value or of a stated limit of a range.

In the present disclosure the term “substantially” can allow for a degree of variability in a value or range, for example, within 80%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage regimen used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA).

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA), wherein said method further comprises a step of adding polyaspartic acid and/or polyglutamic acid for attenuating lung toxicity caused by said polymyxins.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein said therapeutic agent is for the treatment of a respiratory infection.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein said therapeutic agent is delivered by inhalation.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein said therapeutic agent is polymyxin B, polymyxin E (colistin), a polymyxin-like lipopeptide or their salts.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein said polyaspartic acid or polyglutamic acid is from the group consisting of polyaspartic acid, polyglutamic acid or their salts.

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein one or more additional active ingredients are added to the co-administration of polymyxin with polyaspartic acid (PAA) and/or polyglutamic acid (PGA).

In some illustrative embodiments, this disclosure relates to a method for reducing the toxicity of polymyxins as a therapeutic agent comprising a step of co-administration polymyxin with polyaspartic acid (PAA) and/or polyglutamic acid (PGA) as disclosed herein, wherein molar ratio of polymyxin:polyaspartic acid and/or polyglutamic acid ranges from about 1:1 to about 1:20.

In some other illustrative embodiments, this disclosure relates to a method for co-formulating polyaspartic acid and/or polyglutamic acid with a polymyxin.

In some other illustrative embodiments, this disclosure relates to a method for co-formulating polyaspartic acid and/or polyglutamic acid with a polymyxin as disclosed herein, wherein the molar ratio of polymyxin:polyaspartic acid and/or polyglutamic acid ranges from about 1:1 to about 1:20.

Yet in some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said solution or suspension is for inhalation.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said solution or suspension is for the treatment of respiratory infection by inhalation.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said mixed solution or suspension comprises the molar ratio of polymyxin:polyaspartic acid and/or polyglutamic acid ranges from approximately 1:1 to approximately 1:20.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said solution or suspension comprises approximately 5 to 300 mg per milliliter of said polymyxin and polyaspartic acid and/or polyglutamic acid at a molar ratio of approximately 1:1 to approximately 1:20.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said solution or suspension composition comprises polymyxin and polyaspartic acid and/or polyglutamic acid at a molar ratio of approximately of approximately 1:1 to approximately 1:20.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said polymyxins are selected from the group consisting of polymyxin B, colistin, or a polymyxin-like lipopeptide.

In some other illustrative embodiments, this disclosure relates to a process for manufacturing a solution or suspension for nebulization of a polymyxin and polyaspartic acid and/or polyglutamic acid comprising the step of dissolving or suspending polymyxin and polyaspartic acid and/or polyglutamic acid in an aqueous or organic medium to afford said drug solution or suspension as disclosed herein, wherein said polyaspartic acid or polyglutamic acid is from the group consisting of polyaspartic acid, polyglutamic acid or a salt thereof.

In yet some other illustrative embodiments, this disclosure relates to a process for manufacturing a dry powder composition of polymyxin and polyaspartic acid and/or polyglutamic acid.

In yet some other illustrative embodiments, this disclosure relates to a process for manufacturing a dry powder composition of polymyxin and polyaspartic acid and/or polyglutamic acid as disclosed herein, wherein the dry powder is for inhalation.

In yet some other illustrative embodiments, this disclosure relates to a process for manufacturing a dry powder composition of polymyxin and polyaspartic acid and/or polyglutamic acid as disclosed herein, wherein the dry powder is for the treatment of respiratory infection by inhalation.

In yet some other illustrative embodiments, this disclosure relates to a process for manufacturing a dry powder composition of polymyxin and polyaspartic acid and/or polyglutamic acid as disclosed herein, wherein a polymyxin and polyaspartic acid and/or polyglutamic acid are at a molar ratio of approximately of approximately 1:1 to approximately 1:20.

In yet some other illustrative embodiments, this disclosure relates to a process for manufacturing a dry powder composition of polymyxin and polyaspartic acid and/or polyglutamic acid, wherein said polymyxins are selected from the group consisting of polymyxin B, colistin, a polymyxin-like lipopeptide or a salt thereof.

In yet some other illustrative embodiments, this disclosure relates to a process for manufacturing a dry powder composition of polymyxin and polyaspartic acid and/or polyglutamic acid, wherein said polyaspartic acid or polyglutamic acid is from the group consisting of polyaspartic acid, polyglutamic acid or a salt thereof.

In yet some other illustrative embodiments, this disclosure relates to a pharmaceutical composition comprising a product manufactured according to the process as disclosed herein together with one or more pharmaceutically acceptable excipients.

In yet some other illustrative embodiments, this disclosure relates to a method for treating a subject with an infection comprising the step of administrating a therapeutically effective amount of a pharmaceutical composition as disclosed herein.

Polymyxin is a group of antibiotics and the most commonly known are polymyxin B and polymyxin E (also known as Colistin). Colistin is an antibiotic medication used as a last-resort treatment for multidrug-resistant Gram negative infections including pneumonia. These may involve bacteria such as Pseudomonas aeruginosa, Klebsiella pneumoniae, or Acinetobacter baumannii. It comes in a form which can be injected into a vein or into a muscle, or inhaled, known as colistimethate sodium and one which is applied to the skin or taken by mouth, known as colistin sulfate. Resistance to colistin is beginning to appear as of 2017.

Polyaspartic acid and polyglutamic acid are biodegradable and water-soluble polymers which are used in the controlled release of drugs due to its ability to form a hydrophilic network. ^{12,13} Polyaspartic acid can efficiently reduce biofilm formation and bacterial burden of Pseudomonas aeruginosa infection in combination with DNase and colistin. ^{14,15} Polyaspartic acid can also inhibit oxidative stress and heavy metal induced toxicity in zebra fish. ¹⁶ Besides appearing as a promising biomaterial for the delivery of cytotoxic drugs in clinical trials due to nontoxic, biocompatible, and nonimmunogenic qualities, the concentration and time-dependent antimicrobial effect of polyglutamic acid have been reported recently. ^{13,17} Here we have shown that both polyaspartic acid and polyglutamic are potent additives that can significantly reduce polymyxin-induced toxicity in lung. We also developed dry powder co-formulations of polymyxins with polyaspartic acid for pulmonary delivery using spray drying technology and examined their physico-chemical properties.

### EXPERIMENTAL

Materials

Colistin sulfate (MW 1407.67) and polymyxin B sulfate (MW 1441.69) were purchased from BetaPharma Co. Ltd (Wujiang City, JiangSu Province, China). Polyaspartic acid (MW 2,000-11,000) and polyglutamic acid (>750,000) was purchased from Sigma Aldrich (St Louis, MO, USA).

In Vitro Toxicities

Human lung epithelial cells (A549 cells) were obtained from the American Type Culture Collection (ATCC® CCL-185™, Manassas, VA, USA), grown and sub-cultured in Dulbecco's modified Eagle's medium (DMEM) supplemented with 10% fetal bovine serum (FBS). A549 cells (1×10⁵ cells/mL, 12-well plate) were seeded in supplemented DMEM at 37° C. in a humidified atmosphere containing 5% CO₂ until 70% confluency. All experiments were conducted in three replicates. Cells were treated with 1.0 mM polymyxin B in the presence and absence of polyaspartic acid (0.25, 1.0 and 5.0 mM); and cell viability were examined at 24 h with flow cytometry after staining with propidium iodide. The drug concentrations for different formulations were selected based on our previous results on polymyxin toxicity in human lung epithelial cells. ¹⁸ To assess the protective effect of polyglutamic acid, A549 cells (0.5×10⁵ cells/mL, 200 μL/well) were seeded on 96-well plates (#353072, Corning, USA) for overnight at 37° C. Cells were treated with 1.0 mM polymyxin B in the presence and absence of polyglutamic acid (2.5, 5.0 and 10.0 μM). Cell viability was determined at 24 h using propidium iodide staining using EVOS® FL Auto Imaging System (Invitrogen; Approximate fluorescence excitation/emission maxima: 535/617 nm). Nuclei were stained with Hoechst 33342 (Invitrogen; excitation and emission wavelengths of 405 and 410 to 551 nm, respectively) to allow cell counting. Three random fields containing at least 50 cells per treatment condition were analyzed.

The attenuation of mitochondrial superoxide formation and loss of mitochondrial membrane potential caused by polymyxin B were determined. ¹⁸ A549 cells (0.5×10⁵ cells/mL) were grown on 8-well chamber slides (170±5 m polymer coverslip, ibidi, Germany) for overnight at 37° C. Cells were treated with 1.0 mM polymyxin B in the presence and absence of polyaspartic acid (0.25, 1.0 and 5.0 mM). To assess the formation of mitochondrial superoxide and loss of mitochondrial membrane potential MitoSOX Red dye (Invitrogen; excitation and emission wavelengths of 514 and 531 to 622 nm, respectively) and TMRE (Invitrogen; excitation and emission wavelengths of 561 and 568 to 690 nm, respectively) were employed, respectively. Nuclei were stained with Hoechst 33342 (Invitrogen; excitation and emission wavelengths of 405 and 410 to 551 nm, respectively) to allow cell counting. Fluorescence intensity was quantified by confocal laser scanning microscopy (Leica SP8 inverted microscope equipped with a 63× oil immersion objective). The average fluorescence intensity per cell for each treatment was calculated using ImageJ (45, 46). Three random fields containing at least 50 cells per treatment condition were analyzed and the TMRE fluorescence in untreated control cells was set as 100%.

Inhibition of cellular uptake of polymyxin B (1.0 mM) by polyaspartic acid (0.25, 1.0 and 5.0 mM) in A549 was examined at 24 h. ¹⁹ For immunostaining, mouse anti-polymyxin B IgM MAb (1:500 in blocking buffer) and goat anti-mouse IgM conjugated with Alexa Fluor 647 (1:500 in blocking buffer) were employed. Cells nuclei were stained with Hoechst 33342 (2 μg/mL). A Leica SP8 inverted confocal microscope (Leica, Wetzlar, Germany) was used for fluorescence imaging with a 63× oil immersion objective (numerical aperture [NA] 1.4).

In Vivo Toxicity

The animal study was approved by the Monash Animal Ethics Committee, Monash University (Victoria, Australia). All experiments were conducted in accordance with the Australian Code of Practice for the Care and Use of Animals for Scientific Purposes. Swiss Albino mice (female; age, 6 weeks; body weight, 20 to 25 g) were obtained from the Monash Animal Research Platform (Clayton, Victoria, Australia). The animal facility had a 12-h light and 12-h dark cycle, and the temperature and humidity were controlled. Animals were housed individually in metabolic cages and had free access to food and water. Mice were intratracheally administered 10 mg/kg of polymyxin B, 120 mg/kg of polyaspartic acid and the combinations of polyaspartic acid with polymyxin B three times a day.

Histopathological examinations of lungs were performed to assess the pulmonary toxicity by polymyxin B. At 24 h following pulmonary administration, mice were humanely killed and lungs were harvested and fixed in formalin immediately. To avoid any potential artificial damage to the lung epithelium, bronchoalveolar lavage and cardiac puncture were not performed. A semiquantitative scoring (SQS) system was employed to quantify the extent of lung damages. ²⁰ Briefly, we used the following grades for the severity and nature of the histopathological changes in the lungs: grade 0, no changes or mild changes considered insignificant; grade 1, minimal lesions affecting 1 to 25% of the area; grade 2, multifocal lesions affecting 25 to 50% of the area; and grade 3, severe tissue changes affecting >50% of the area. The grades were given the following scores: grade 0, 0.1; grade 1, 1; grade 2, 4; and grade 3, 10. Percentages of damage across different levels of the lungs were given the following scores: <1%, 0; from 1 to <5%, 1; from 5 to <10%, 2; from 10 to <20%, 3; from 20 to <30%, 4; from 30 to <40%, 5; >40%, 6. The overall lung histology score was determined as the product of the grade and percent damage score. An SQS was assigned the following scores: SQS 0, no significant change; SQS+1, mild damage; SQS+2, mild to moderate damage; SQS+3, moderate damage; SQS+4, moderate to severe damage; and SQS+5, severe damage.

Spray Drying

Spray-dried formulations (Table 1) were prepared by spray drying (SD) aqueous solutions (20 mg/mL of total solids) of single pure component or combination formulations of polyaspartic acid (PAA) and colistin (Col) using a BUCHI B-290 mini spray dryer with a standard two-fluid nozzle (BUCHI Labortechnik AG, Flawil, Switzerland). Spray drying was conducted at a feed rate of 2 mL/min with an inlet air temperature (Tᵢₙ) of 120±5° C., aspirator at 40 kg/h (100%), and an atomizing air pressure of 60 mm Hg. The spray-dried product containing polyaspartic acid was stored in a refrigerator at −20° C., while spray dried pure colistin was stored in a desiccator containing silica gel to maintain 20±2% RH at 20±2° C.

TABLE 1
Compositions of the spray-dried formulations

| Formulation | Components | Molar ratio | Weight ratio |
|---|---|---|---|
| SD Col | Col | 1 | 1.00 |
| | PAA | 0 | 0.00 |
| SD 5:1 Col-PAA | Col | 5 | 1.00 |
| | PAA | 1 | 1.02 |
| SD 1:1 Col-PAA | Col | 1 | 1.00 |
| | PAA | 1 | 5.12 |
| SD 1:5 Col-PAA | Col | 1 | 1.00 |
| | PAA | 5 | 25.6 |
| SD PAA | Col | 0 | 0.00 |
| | PAA | 1 | 1.00 |

Particle Size Distribution (PSD)

The PSD of fresh formulations was determined using a Mastersizer 3000 equipped with Aero-S for dry powder dispersion (Malvern Instruments, Worcestershire, UK). This instrument is based on laser diffraction technique and thus provides a volume distribution of particle size. Powder sample was fed into the dispersion system for analysis. Each formulation was measured using three independent replicates. The average percentile diameters D₁₀, D₅₀, and D₉₀ were obtained and used to characterize the particle size distribution. The width of the distribution was determined using ‘span’, which is calculated as the ratio of (D₉₀−D₁₀) to D₅₀. Smaller span values represent narrower particle size distributions.

Scanning Electron Microscopy (SEM)

The particle morphology of samples was examined by SEM (NOVA nanoSEM, FEI Company, Hillsboro, Oregon, USA). The powder sample was dispersed on a sample stub mounted with adhesive carbon tape. The overlapping particles were removed using pressurized air and then coated with a thin platinum film using a sputter coater (208 HR, Cressington Sputter Coater, England, UK). The coated samples were then mounted on the SEM stage and observed.

Powder X-Ray Diffraction (PXRD)

A Rigaku Smartlab™ diffractometer (Rigaku Americas, The Woodlands, TX) with a Cu-Kα radiation source was used to evaluate powder crystallinity. The diffraction patterns were recorded at 2θ 4 to 400 and a scan speed of 4°/min. The radiation source was operated at 40 kV voltage and 44 mA current.

In Vitro Aerosolization Performance

A Next-Generation Impactor (NGI) was used to determine the in vitro aerosolization performance of colistin-polyaspartic acid formulations (NGI, Copley, Nottingham, UK). The powder sample (10±1 mg) was filled in size-3 HPMC capsules (Qualicaps, Whitsett, NC). A filled capsule was loaded in a low-resistance RS01 DPI device (Plastiape S.p.A., Osnago, Italy) and pierced. After attaching the device to the NGI assembly, four liters of air was drawn through the inhaler by a vacuum pump operated at 100 L/min for 2.4 s. This corresponds to a pressure drop of ˜4 kPa across the RS01 DPI device. For each replicate, four capsules were dispersed by loading four 10-mg capsules. Four replicates were analyzed for each formulation. The dispersed powder was collected from the capsule, the device, the NGI throat and different NGI stages by washing with water. The drug content was analyzed using HPLC to determine the amount of colistin collected from each location. Emitted dose (ED) was measured as the percentage of drug that was released from the device. Fine particle fraction (FPF) was calculated as the percentage of drug present in particles with an aerodynamic diameter<5 μm. Finally, the percentage of emitted particles with an aerodynamic size below 5 μm—also called the emitted FPF (E-FPF)—was calculated from ED and FPF.

Analytical Method for Colistin

Colistin concentration was measured by an established high-performance liquid chromatography (HPLC) method which is described here briefly. An Agilent 1260 HPLC system (Agilent Technologies, Santa Clara, CA) and an Eclipse Plus C18 column (5 μm, 150×4.6 mm, Agilent, Waldbronn, Germany) were used. A mobile phase composed of 76% (v/v) 30 mM sodium sulfate solution (adjusted to pH 2.5 with H₃PO₄), and 24% (v/v) acetonitrile was pumped at an isocratic flow rate of 1.0 mL/min. The injection volume was 30 μL and the column was operated at room temperature (˜21° C.). Colistin was detected at a wavelength of 214 nm.

Statistical Analysis

Results are expressed as mean±standard deviation (SD) and an Analysis of Variance (ANOVA) test was performed for statistical comparisons of multiple formulations.

Results

Attenuation of Polymyxin-Induced Lung Toxicity In Vitro and In Vivo

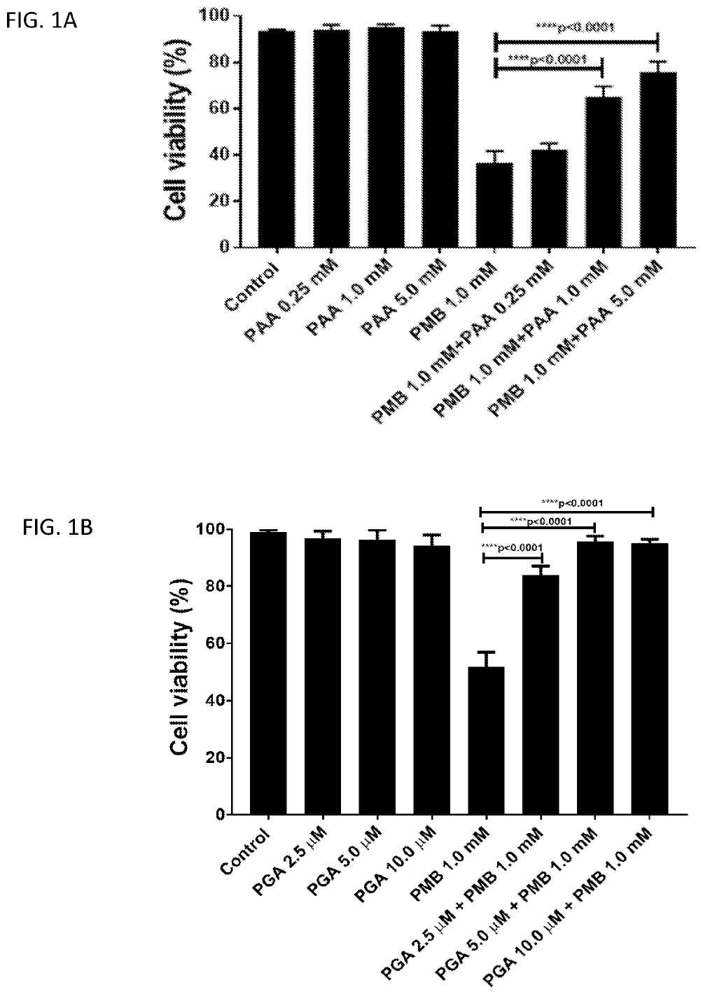

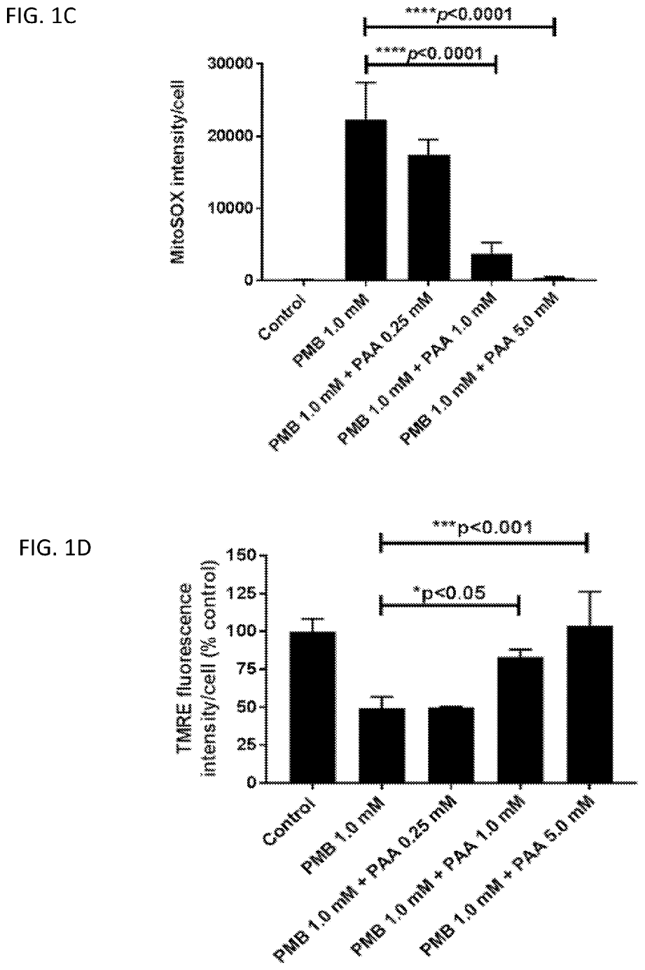

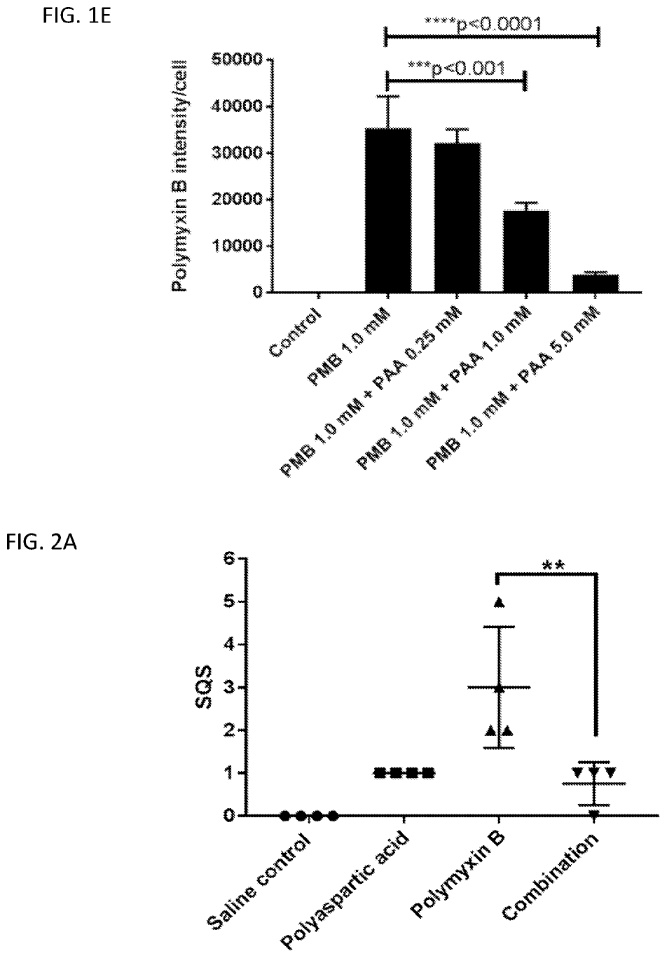

Both polyaspartic acid and polyglutamic acid significantly attenuated polymyxin B induced toxicity in A549 cells in a concentration-dependent manner (FIG. 1A-E). Cell viability reduced to 36.7±5.1% with 1.0 mM polymyxin B treatment at 24 h, which was significantly increased to 65.2±4.5% (p<0.0001) and 75.7±4.7% (p<0.0001) when cells were pre-treated of 45 min with 1.0 mM and 5.0 mM of polyaspartic acid, respectively (FIG. 1A). Similarly, at 24 h the cell viability was 51.7±5.2% after 1.0 mM polymyxin B treatment which was increased up to 83.87±3.1% (p<0.0001), 95.33±1.5% (p<0.0001) and 95.77±1.5% (p<0.0001) following pre-treatment of 45 min with 2.5 μM, 5.0 μM and 10.0 μM of polyglutamic acid, respectively (FIG. 1B).

At 24 h, MitoSOX fluorescence intensity in the A549 cells treated with 1.0 mM polymyxin B alone was >200-fold higher (p<0.0001) compared to the control (FIG. 1C). When concomitantly treated with 1.0 mM and 5.0 mM of polyaspartic acid, oxidative stress induced by 1.0 mM of polymyxin B was reduced by 37-fold (p<0.0001) and 3.7-fold (p<0.0001), respectively, compared to the control. Polyaspartic acid (0.25 mM) also reduced polymyxin-induced oxidative stress to some extent but was not statistically significant (p>0.05). Mitochondrial membrane potential was reduced to <50% (49.5±7.2%) in A549 cells after treatment with 1.0 mM of polymyxin B compared to the control but was increased significantly to 83.3±4.8% (p<0.05) and 103.5±22.6% (p<0.001) when concurrently treated with 1.0 mM and 5.0 mM polyaspartic acid, respectively (FIG. 1D).

Furthermore, the immunofluorescence intensity of polymyxin B in A549 cells indicates the inhibition of intracellular uptake of polymyxin B by >2-fold (p<0.001) and >9-fold (p<0.0001) in the presence of 1.0 and 5.0 mM polyaspartic acid, respectively (FIG. 1E).

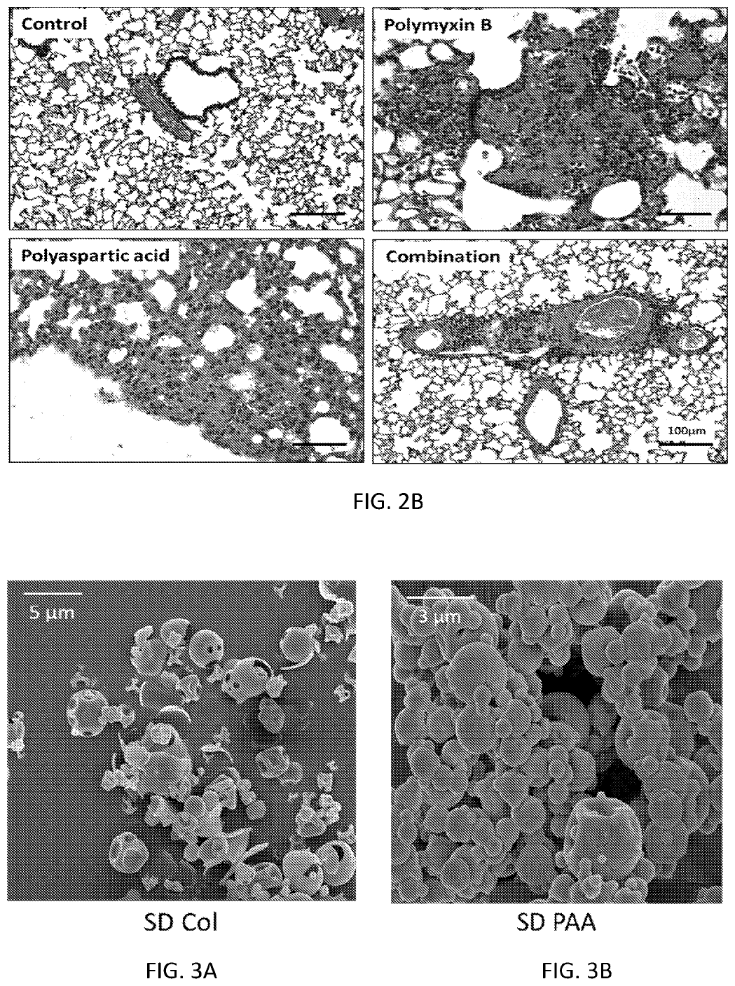

Histological data confirmed that polyaspartic acid significantly reduced polymyxin B-induced pulmonary toxicity in vivo (p****<0.0001) (FIGS. 2A and 2B). Control (0.9% saline) group showed no macroscopic and microscopic lesion (SQS: 0); 10 mg/kg polymyxin B showed no macroscopic lesion but severe tissue injury affecting 25-50% of the area (SQS: +2 to +5); 120 mg/kg polyaspartic acid showed minimal to mild damage affecting <25% of the area with mild focal intra-alveolar haemorrhage (SQS: +1); while the combination showed minimal damage with perivascular inflammation (SQS: +1) of the tissue (FIG. 2B). Overall semi-quantitative score (SQS) of lung damage was 3.0±1.4 in the polymyxin B treated group, which was reduced to 0.75±0.5 (n=4) with co-administration of polyaspartic acid (FIG. 2A).

Particle Morphology and Size Distribution

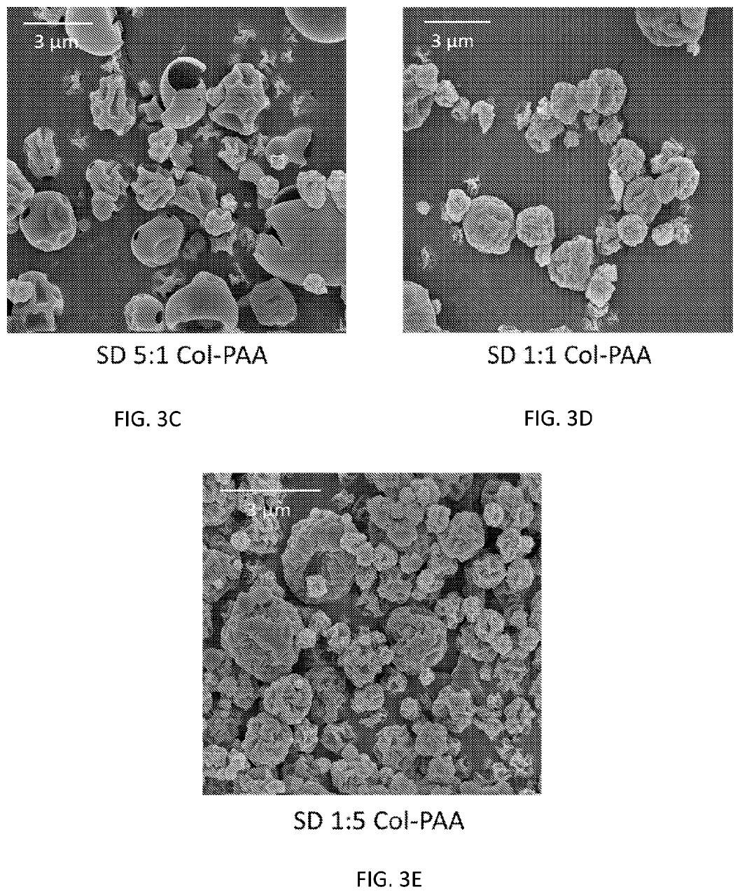

FIG. 3 shows the representative scanning electron microscopy (SEM) images of spray dried pure and combinations formulations of colistin (Col) and polyaspartic acid (PAA). Spray-dried (SD) colistin particles formed either dimpled spheres or hollow smooth shells with fractures or holes. The formulation with a higher proportion of colistin—5:1 col-PAA—resembled pure SD Col in morphological features and variety. The formulations with a higher proportion of PAA—both 1:1 and 1:5 col-PAA—showed spheroidal particles with highly wrinkled surfaces. Pure SD PAA showed smooth spherical particles that had fused together to form large clusters. These particles also had hair-line cracks on their surface, likely due to charging during imaging.

The particle size distribution of different formulations is summarized in Table 2. Approximate 80% of the particles for pure SD col and all combination formulations lay between 1 and 5 μm. This shows that the particles are in the optimal size range for use as dry powder inhalers ²¹. Pure SD PAA showed a remarkably high D₉₀ (329 μm) while its D₁₀ and D₅₀ values are similar to those of other formulations. This indicates formation of large agglomerates by micron-sized particles which was also shown by SEM images.

TABLE 2
Particle size distribution summary of
different spray-dried formulations

| Samples | | D₁₀ | D₅₀ | D₉₀ | Span |
|---|---|---|---|---|---|
| SD Col | Average | 1.13 | 2.39 | 5.80 | 2.0 |
| | S.D. | 0.01 | 0.03 | 0.38 | 0.2 |
| SD 5:1 Col-PAA | Average | 1.08 | 2.29 | 4.66 | 1.57 |
| | S.D. | 0.01 | 0.00 | 0.10 | 0.05 |
| SD 1:1 Col-PAA | Average | 0.99 | 2.14 | 4.85 | 1.81 |
| | S.D. | 0.02 | 0.03 | 0.09 | 0.02 |
| SD 1:5 Col-PAA | Average | 0.97 | 2.00 | 4.42 | 1.72 |
| | S.D. | 0.01 | 0.04 | 0.50 | 0.21 |
| SD PAA | Average | 1.08 | 4.46 | 329 | 75.7 |
| | S.D. | 0.04 | 0.89 | 25.6 | 10.9 |

Crystallinity

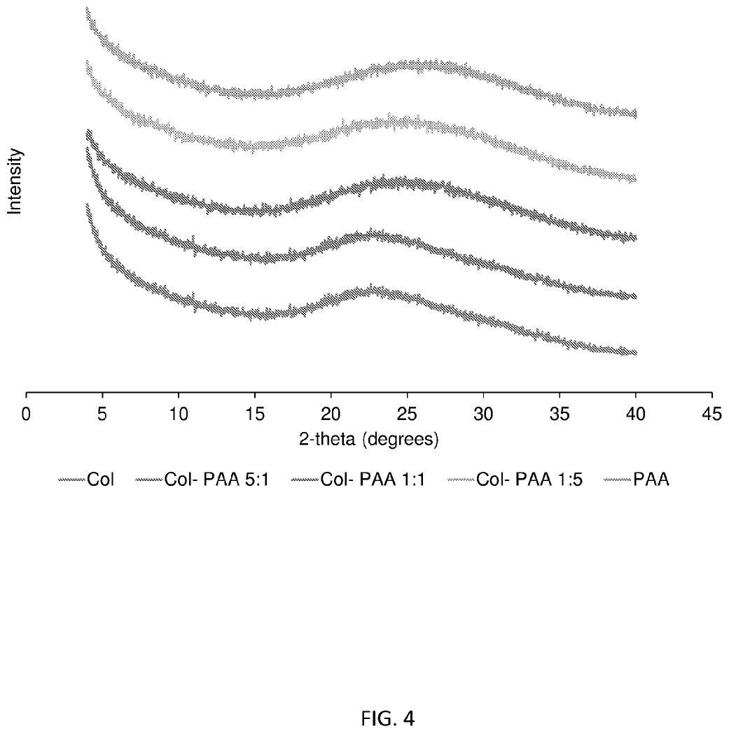

The powder X-ray diffraction pattern of pure and combination formulations has been shown in FIG. 4. All formulations showed no crystallinity peaks which shows that they are amorphous.

In-Vitro Aerosolization Performance

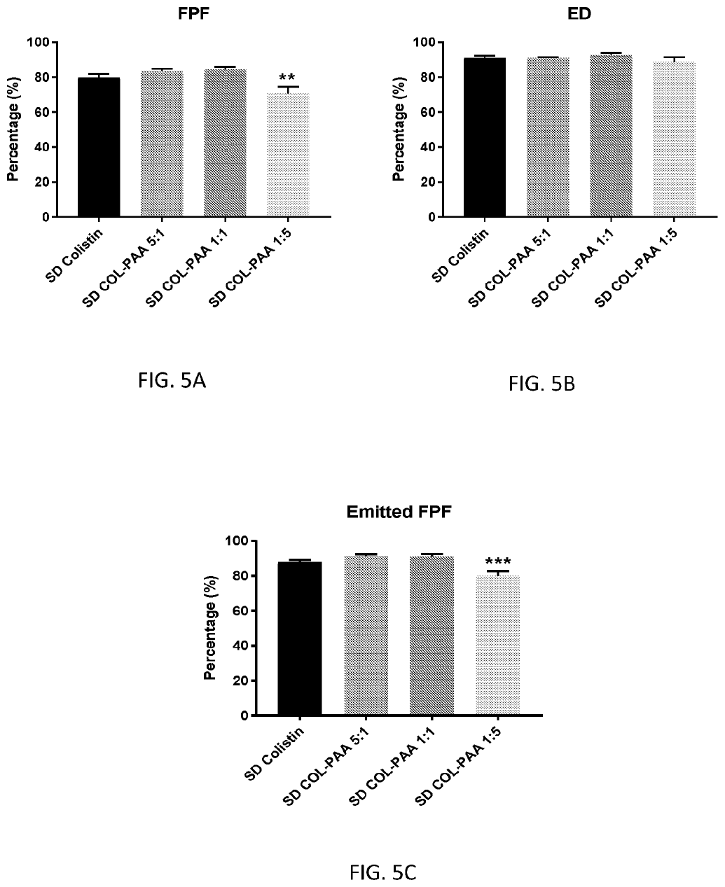

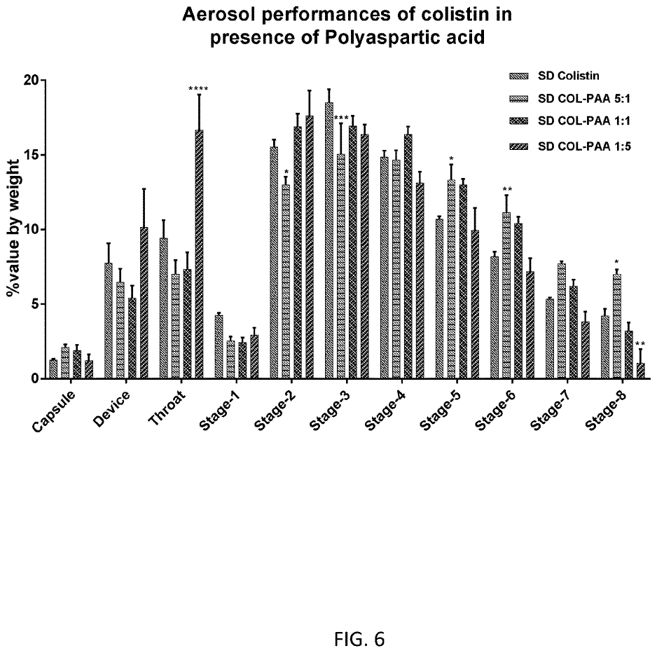

FIGS. 5 and 6 show the fine particle fraction (FPF), emitted dose (ED), emitted FPF (E-FPF) and NGI deposition profiles of different col-PAA spray-dried formulations. Pure colistin and all combinations 5:1, 1:1 and 1:5 showed ED values in the range of 88-93%. The ED values of all formulations were similar. On the other hand, a significantly lower FPF of 71% was observed for the 5:1 Col-PAA formulation relative to pure colistin and other combinations. This trend was reflected in the E-FPF values where pure colistin, 5:1, and 1:1 coformulations attained close to 90% while 1:5 Col-PAA scored only 80%. Although not significant, the ED, FPF and E-FPF values increased slightly as the proportion of PAA in the formulation increased when comparing pure colistin (0% PAA by wt.), 5:1 (50% PAA) and 1:1 col-PAA (84% PAA) combinations.

CONCLUSIONS. We have discovered that polyaspartic acid and polyglutamic acid inhibit polymyxin-induced toxicities (including cell death, production of mitochondrial superoxide and loss of mitochondrial membrane potential) in human lung epithelial cells in a dose-dependent manner. Co-administration of polyaspartic acid significantly attenuates lung toxicity induced by polymyxins in mice. All dry powder formulations of colistin and polyaspartic acid combinations have inhalable particle size distributions and an amorphous solid form. The 1:5 Col-PAA formulation containing approximately 96% polyaspartic acid (w/w) shows poorer dispersion with significantly lower FPF (71%) and E-FPF (˜80%) value compared to other formulations. Combination formulations 5:1 and 1:1 col-PAA showed excellent fine particle fraction (>80%) and emitted dose (>90%) values and were similar to that of pure SD col. Our data demonstrate that adding colistin in the formulation improves FPF.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

1. Velkov T, Abdul Rahim N, Zhou Q T, Chan H K, Li J 2015. Inhaled anti-infective chemotherapy for respiratory tract infections: successes, challenges and the road ahead. Adv Drug Deliv Rev 85:65-82.
2. Biagi M, Butler D, Tan X, Qasmieh S, Wenzler E 2019. A Breath of Fresh Air in the Fog of Antimicrobial Resistance: Inhaled Polymyxins for Gram-Negative Pneumonia. Antibiotics-Basel 8(1).
3. Conway S P 2005. Nebulized antibiotic therapy: the evidence. Chron Respir Dis 2(1):35-41.
4. Hoiby N, Frederiksen B, Pressler T 2005. Eradication of early Pseudomonas aeruginosa infection. J Cyst Fibros 4 Suppl 2:49-54.
5. Leong K W, Ong S, Chee H L, Lee W, Kwa A L 2010. Hypersensitivity pneumonitis due to high-dose colistin aerosol therapy. International journal of infectious diseases: IJID: official publication of the International Society for Infectious Diseases 14(11):e1018-1019.
6. Lepine P A, Dumas A, Boulet L P 2017. Pulmonary Eosinophilia From Inhaled Colistin. Chest 151(1):e1-e3.
7. McCoy K S 2007. Compounded colistimethate as possible cause of fatal acute respiratory distress syndrome. The New England journal of medicine 357(22):2310-2311.
8. Wahby K, Chopra T, Chandrasekar P 2010. Intravenous and Inhalational Colistin-Induced Respiratory Failure. Clinical Infectious Diseases 50(6):e38-e40.
9. Ahmed M U, Velkov T, Lin Y-W, Yun B, Nowell C J, Zhou F, Zhou Q T, Chan K, Azad M A K, Li J 2017. Potential Toxicity of Polymyxins in Human Lung Epithelial Cells. Antimicrobial agents and chemotherapy 61(6):e02690-02616.
10. Ahmed M U, Velkov T, Zhou Q T, Fulcher A J, Callaghan J, Zhou F, Chan K, Azad M A K, Li J 2018. Intracellular localization of polymyxins in human alveolar epithelial cells. Journal of Antimicrobial Chemotherapy 74(1):48-57.
11. Ahmed M U, Azad M A K, Chan K, Wang J, Zhou F, Chan K, Zhou Q T, Velkov T, Li J 2018. Polyaspartic acid inhibits polymyxin B induced pulmonary toxicity (01031). 28th European congress of clinical microbiology & infectious diseases (ECCMID), Madrid Spain.
12. Sharma S, Dua A, Malik A 2014. Polyaspartic acid based superabsorbent polymers. European Polymer Journal 59:363-376.
13. Manocha B, Margaritis A 2008. Production and characterization of gamma-polyglutamic acid nanoparticles for controlled anticancer drug release. Crit Rev Biotechnol 28(2):83-99.
14. Bucki R, Cruz K, Pogoda K, Eggert A, Chin L, Ferrin M, Imbesi G, Hadjiliadis D, Janmey P A 2015. Enhancement of Pulmozyme activity in purulent sputum by combination with poly-aspartic acid or gelsolin. Journal of cystic fibrosis: official journal of the European Cystic Fibrosis Society 14(5):587-593.
15. Robertson D M, Parks Q M, Young R L, Kret J, Poch K R, Malcolm K C, Nichols D P, Nichols M, Zhu M, Cavanagh H D, Nick J A 2011. Disruption of contact lens-associated Pseudomonas aeruginosa biofilms formed in the presence of neutrophils. Investigative ophthalmology & visual science 52(5):2844-2850.
16. Liu T, Wang R, Cao H, Lin A 2017. Polyaspartic acid alleviates heavy metal toxicity in zebrafish (Danio rerio). Chemistry and Ecology 33(7):684-693.
17. Ajayeoba T A, Dula S, Ijabadeniyi O A 2019. Properties of Poly-gamma-Glutamic Acid Producing-Bacillus Species Isolated From Ogi Liquor and Lemon-Ogi Liquor. Front Microbiol 10:771.
18. Ahmed M U, Velkov T, Lin Y W, Yun B, Nowell C J, Zhou F, Zhou Q T, Chan K, Azad M A K, Li J 2017. Potential toxicity of polymyxins in human lung epithelial cells. Antimicrob Agents Chemother 61(6).
19. Ahmed M U, Velkov T, Zhou Q T, Fulcher A J, Callaghan J, Zhou F, Chan K, Azad M A K, Li J 2019. Intracellular localization of polymyxins in human alveolar epithelial cells. J Antimicrob Chemother 74(1):48-57.
20. Lin Y W, Zhou Q, Onufrak N J, Wirth V, Chen K, Wang J, Forrest A, Chan H K, Li J 2017. Aerosolized polymyxin B for treatment of respiratory tract infections: Determination of pharmacokinetic-pharmacodynamic indices for aerosolized polymyxin B against Pseudomonas aeruginosa in a mouse lung infection model. Antimicrob Agents Chemother 61(8).
21. Hoppentocht M. 2015. Dry powder inhalation of antibiotics; a promising approach for treatment of bacterial infections. ed.

1. A method for reducing the toxicity of polymyxins as a therapeutic agent comprising a step of co-administration polymyxin with polyaspartic acid (PAA), polyglutamic acid (PGA), a combination of PAA and PGA, or a pharmaceutically acceptable salt thereof.

2. The method for reducing the toxicity of polymyxins according to claim 1 further comprising a step of adding polyaspartic acid and/or polyglutamic acid for attenuating lung toxicity caused by said polymyxins.

3. The method for reducing the toxicity of polymyxins according to claim 1, wherein said therapeutic agent is for the treatment of a respiratory infection.

4. The method for reducing the toxicity of polymyxins according to claim 1, wherein said therapeutic agent is delivered by inhalation.

5. The method for reducing the toxicity of polymyxins according to claim 1, wherein said therapeutic agent is polymyxin B, polymyxin E (colistin), a polymyxin-like lipopeptide or their salts.

6. The method for reducing the toxicity of polymyxins according to claim 1, wherein said polyaspartic acid or polyglutamic acid is from the group consisting of polyaspartic acid, polyglutamic acid or their salts.

7. The method for reducing the toxicity of polymyxins according to claim 1, with another one or more active ingredients are added to the co-administration of polymyxin with polyaspartic acid (PAA), polyglutamic acid (PGA), a combination of PAA or PGA, or a pharmaceutically acceptable salt thereof.

8. The method for reducing the toxicity of polymyxins according to claim 1, wherein molar ratio of polymyxin:polyaspartic acid and/or polyglutamic acid ranges from about 1:1 to about 1:20.

9. A method for co-formulating poly aspartic acid (PAA), polyglutamic acid (PGA), a combination of PAA and PGA, or a pharmaceutically acceptable salt thereof, together with a polymyxin.

10. The method for co-formulating polyaspartic acid and/or polyglutamic acid with polymyxin according to claim 9, wherein the molar ratio of polymyxin:polyaspartic acid and/or polyglutamic acid ranges from about 1:1 to about 1:20.

11. A process for manufacturing a solution or suspension for nebulization of a Polymyxin and polyaspartic acid and/or polyglutamic acid comprising the step of dissolving or suspending polymyxin and polyaspartic acid (PAA), polyglutamic acid (PGA), a combination of PAA and PGA, or a pharmaceutically acceptable salt thereof in an aqueous or organic medium to afford said drug solution or suspension.

12. The process of claim 11, wherein said solution or suspension is for inhalation.

13. The process of claim 11, wherein said solution or suspension is for the treatment of respiratory infection by inhalation.

14. The process of claim 11, wherein said mixed solution or suspension comprises the molar ratio of polymyxin:polyaspartic acid and/or polyglutamic acid ranges from approximately 1:1 to approximately 1:20.

15. The process of claim 1, wherein said solution or suspension comprises approximately 5 to 300 mg per milliliter of said polymyxin and polyaspartic acid and/or polyglutamic acid at a molar ratio of approximately 1:1 to approximately 1:20.

16. The solution or suspension composition according to claim 11, wherein polymyxin and polyaspartic acid and/or polyglutamic acid are at a molar ratio of approximately 1:1 to approximately 1:20.

17. The solution or suspension composition according to claim 11, wherein said polymyxins are selected from the group consisting of polymyxin B, colistin, or a polymyxin-like lipopeptide.

18. The solution or suspension composition according to claim 11, wherein said polyaspartic acid or polyglutamic acid is from the group consisting of polyaspartic acid (PAA), polyglutamic acid (PGA), a combination of PAA and PGA, or a pharmaceutically acceptable salt thereof.

19. A process for manufacturing a dry powder composition of polymyxin and polyaspartic acid (PAA), polyglutamic acid (PGA), a combination of PAA and PGA, or a pharmaceutically acceptable salt thereof.

20. The process of claim 19, wherein the dry powder is for inhalation.

21. The process of claim 19, wherein the dry powder is for the treatment of respiratory infection by inhalation.

22. The dry powder composition according to claim 19, wherein a polymyxin and polyaspartic acid and/or polyglutamic acid are at a molar ratio of approximately 1:1 to approximately 1:20.

23. The dry powder composition according to claim 19, wherein said polymyxins are selected from the group consisting of polymyxin B, colistin, a polymyxin-like lipopeptide, or a pharmaceutically acceptable salt thereof.

24. The dry powder composition according to claim 19, wherein said polyaspartic acid or polyglutamic acid is from the group consisting of polyaspartic acid (PAA), polyglutamic acid (PGA), a combination of PAA and PGA, or a pharmaceutically acceptable salt thereof.

25. A pharmaceutical composition comprising a product manufactured according to the process of claim 19 together with one or more pharmaceutically acceptable excipients.

26. A method for treating a subject with an infection comprising the step of administrating a therapeutically effective amount of a pharmaceutical composition according to claim 19.
